SEQUENCE LISTING (28 sequences)

SEQ ID NO: 1 - protein, 51 aa, synthetic construct
GIVEQCCTSICSLYQLENYCGGSLPETGGGSLEEEWAQIQSEVWGRGSPSY

SEQ ID NO: 2 - protein, 29 aa, synthetic construct
FVNQHLCGSHLVEALYLVCGERGFFYTPK

SEQ ID NO: 3 - protein, 51 aa, synthetic construct
GIVEQCCTSICSLYQLENYCGGSLPETGGGSLEEEWAQIQCEVWGRGCPSY

SEQ ID NO: 4 - protein, 25 aa, synthetic construct
GGGSLEEEEWAQIQSEVWGRGSPSY

SEQ ID NO: 5 - protein, 21 aa, synthetic construct
GIVEQCCTSICSLYQLENYCN

SEQ ID NO: 6 - protein, 48 aa, synthetic construct
SLEEEWAQIQSEVWGRGSFVNQHLCGSHLVEALYLVCGERGFFYTPKT

SEQ ID NO: 7 - protein, 29 aa, synthetic construct
GIVEQCCTSICSRYQLENYCGGSLPETGG

SEQ ID NO: 8 - protein, 51 aa, synthetic construct
GIVEQCCTSICSRYQLENYCGGSLPETGGGSLEEEWAQIQSEVWGRGSPSY

SEQ ID NO: 9 - protein, 29 aa, synthetic construct
GIVEQCCHRICSLYQLENYCGGSLPETGG

SEQ ID NO: 10 - protein, 29 aa, synthetic construct
FVNQHLCGSELVEALYLVCGERGFFYTPK

SEQ ID NO: 11 - protein, 51 aa, synthetic construct
GIVEQCCHRICSLYQLENYCGGSLPETGGGSLEEEWAQIQSEVWGRGSPSY

SEQ ID NO: 12 - protein, 51 aa, synthetic construct
GIVEQCCTSICSLYQLENYCGGSLPETGGGSLEEEWAQIQSEVWGRGSPDY

SEQ ID NO: 13 - skipped

SEQ ID NO: 14 - protein, 30 aa, synthetic construct
FVNQHLCGSHLVEALYLVCGERGFFYTPKT

SEQ ID NO: 15-17 - skipped (3 entries)

SEQ ID NO: 18 - protein, 27 aa, synthetic construct
GIVEQCCTSICSLYQLENYCGLPETGG

SEQ ID NO: 19 - protein, 28 aa, synthetic construct
GIVEQCCTSICLYQLENYCGGSLPETGG

SEQ ID NO: 20 - protein, 10 aa, synthetic construct
LPETGGGFYK

SEQ ID NO: 21 - protein, 43 aa, synthetic construct
GSLDESFYDWFERQLGGGSGGSGLEEEWAQIQSEVWGRGSPSY

SEQ ID NO: 22 - protein, 25 aa, synthetic construct
GGGSGLEEEWAQIQSEVWGRGSPSY

SEQ ID NO: 23 - protein, 31 aa, synthetic construct
GGSLPETGGGSLEEEWAQIQSEVWGRGSPSY

SEQ ID NO: 24 - protein, 23 aa, synthetic construct
GGGSLEEEWAQIQSEVWGRGSPS

SEQ ID NO: 25 - protein, 18 aa, synthetic construct
GGGGSLDESFYDWFERQL

SEQ ID NO: 26 - protein, 24 aa, synthetic construct
GGGSGSGSWAQIQSEVWGRGSPSY

SEQ ID NO: 27 - protein, 57 aa, synthetic construct
GIVEQCCTSICSLYQLENYCGGSLPETGGGSGGSGGSLEEEWAQIQSEVWGRGSPSY

SEQ ID NO: 28 - protein, 43 aa, synthetic construct
GSLDESFYDWFERQLGGGSGGSSLEEEWAQIQCEVWGRGCPSY

(19) United States
(12) Patent Application Publication
(10) Pub. No.: US 2026/0209304 A1
(43) Pub. Date: Jul. 23, 2026

(54) INSULIN RECEPTOR ANTAGONIST

(71) Applicant: The Board of Trustees of the Leland Stanford Junior University, Stanford, CA (US)

(72) Inventors: Danny Hung-Chieh Chou, Palo Alto, CA (US); Claire Park, Redwood City, CA (US); Nai-Pin Lin, Redwood City, CA (US); Yanxian Zhang, Redwood City, CA (US)

(21) Appl. No.: 19/140,177

(22) PCT Filed: Dec. 20, 2023
(86) PCT No.: PCT/US2023/085155

§ 371 Date: Jun. 17, 2025

Related U.S. Application Data
(60) Provisional application No. 63/433,983, filed on Dec. 20, 2022.

(51) Int. Cl. C07K 14/62; A61K 38/00; A61P 3/10; C07K 14/00

(52) U.S. Cl. CPC C07K 14/62; A61P 3/10; C07K 14/001; A61K 38/00

(57) ABSTRACT

Polypeptide antagonists of the human insulin receptor are provided. In some embodiments, a pharmaceutical formulation is provided, comprising an INSR antagonist as identified herein, and a pharmaceutically acceptable excipient. The formulation may be provided in a unit dose, e.g. a therapeutically effective dose. In methods of the disclosure, an effective dose of a an INSR antagonist as identified herein is administered to an individual having, or at risk of having, hyperinsulinemia, in a dose effective to stabilize, reduce or prevent clinical symptoms of the disease. The individual may be monitored for clinical indicia of disease before, during, and/or after administration.

### CROSS REFERENCE TO OTHER APPLICATIONS

This application claims the benefit of U.S. Provisional Application No. 63/433,983, filed Dec. 20, 2022, the contents of which are hereby incorporated by reference in its entirety.

### INCORPORATION BY REFERENCE OF SEQUENCE LISTING PROVIDED AS AN XML FILE

A Sequence Listing is provided herewith as a Sequence Listing XML, “S22-373_STAN-2028WO_SEQ_LIST.xml” created on Oct. 6, 2023 and having a size of 202,752 bytes. The contents of the Sequence Listing XML are incorporated by reference herein in their entirety.

### BACKGROUND

Insulin initiates the regulation of cellular glucose metabolism by binding to the insulin receptor (INSR) on the cell surface, a process that activates the receptor's intrinsic kinase activity. When activated, the INSR undergoes autophosphorylation, followed by the recruitment and phosphorylation of INSR signaling molecules, including the IRS proteins and members of the phosphotidylinositol 3-kinase (PI3K)-Akt pathway. In cells, activation of this pathway by insulin results in the translocation of glucose transporters to the cell surface with subsequent uptake of glucose.

Hypoglycemia due to insulin excess from both exogenous and endogenous sources is not an infrequent clinical condition. In some instances, the current treatments for insulin-induced hypoglycemia do not adequately restore normoglycemia, resulting in prolonged hospitalization or neurological damage. For example, diabetic patients treated with either insulin or insulin-releasing agents can develop symptomatic hypoglycemia. This hypoglycemia can be severe and sustained, requiring visits to the emergency department and or prolonged hospitalization. Inhibition of INSR signaling in vivo can be useful for the short-term treatment of hypoglycemia caused by exogenous insulin.

In addition, rare conditions of sustained endogenous hyperinsulinemia can occur, where current therapies are not always adequate. These conditions include insulinoma, excess secretion of IGF-II and congenital hyperinsulinemia (CHI). Congenital hyperinsulinism (HI) is a genetic disorder of pancreatic beta-cell function characterized by failure to suppress insulin secretion in the setting of hypoglycemia, resulting in severe hypoglycemia that can cause brain damage or death if inadequately treated. Roughly 50% of the patients can use drugs to manage but the other half will need to undergo pancreatectomy, which will lead to long term complications down the road.

Therapies that attenuate insulin signaling via inhibition of the INSR would be effective for the treatment of sustained and life threatening hyperinsulinemic hypoglycemia. The present disclosure addresses this need.

### SUMMARY

Compositions and methods are provided for inhibition of insulin receptor signaling. The compositions provided are antagonists of the human insulin receptor, referred to here as an INSR antagonist, having the structure of a polypeptide heterodimer with a sequence:

SEQ ID NO:1 and SEQ ID NO:2, disulfides: SEQ ID NO:1 residue C6 and SEQ ID NO: 1 residue C11; SEQ ID NO:1 residue C7 and SEQ ID NO:2 residue C7; SEQ ID NO:1 residue C20 and SEQ ID NO:2 residue C19;

SEQ ID NO:3 and SEQ ID NO:2, disulfides: SEQ ID NO:3 residue C6 and SEQ ID NO: 3 residue C11; SEQ ID NO:3 residue C41 and SEQ ID NO:3 residue C48; SEQ ID NO:3 residue C7 and SEQ ID NO:2 residue C7, SEQ ID NO:3 residue C20 and SEQ ID NO:2 residue C19;

SEQ ID NO:27 and SEQ ID NO:2, disulfides: SEQ ID NO:27 residue C6 and SEQ ID NO: 27 residue C11; SEQ ID NO:27 residue C7 and SEQ ID NO:2 residue C7; SEQ ID NO:27 residue C20 and SEQ ID NO:2 residue C19;

SEQ ID NO:5 and SEQ ID NO:6, disulfides: SEQ ID NO:5 residue C6 and SEQ ID NO: 5 residue C11, SEQ ID NO:5 residue C7 and SEQ ID NO:6 residue C25, SEQ ID NO:5 residue C20 and SEQ ID NO:6 residue C37;

SEQ ID NO:7 and SEQ ID NO:2, disulfides: SEQ ID NO:7 residue C6 and SEQ ID NO: 7 residue C11, SEQ ID NO:7 residue C7 and SEQ ID NO:2 residue C7, SEQ ID NO:7 residue C20 and SEQ ID NO:2 residue C19;

SEQ ID NO:8 and SEQ ID NO:2, disulfides: SEQ ID NO:8 residue C6 and SEQ ID NO: 8 residue C11, SEQ ID NO:8 residue C7 and SEQ ID NO:2 residue C7, SEQ ID NO:8 residue C20 and SEQ ID NO:2 residue C19;

SEQ ID NO:9 and SEQ ID NO:10, disulfides: SEQ ID NO:9 residue C6 and SEQ ID NO: 9 residue C11, SEQ ID NO:9 residue C7 and SEQ ID NO:10 residue C7, SEQ ID NO:9 residue C20 and SEQ ID NO:10 residue C19;

SEQ ID NO:11 and SEQ ID NO:10, disulfides: SEQ ID NO:11 residue C6 and SEQ ID NO: 11 residue C11, SEQ ID NO:11 residue C7 and SEQ ID NO:10 residue C7, SEQ ID NO:11 residue C20 and SEQ ID NO:10 residue C19; and

SEQ ID NO:12 and SEQ ID NO:2, disulfides: SEQ ID NO:12 residue C6 and SEQ ID NO: 12 residue C11, SEQ ID NO: 12 residue C7 and SEQ ID NO:2 residue C7, SEQ ID NO: 12 residue C20 and SEQ ID NO:2 residue C19.

The provided insulin receptor antagonists are demonstrated to have a dose-dependent inhibition of native insulin activation of human INSR.

In some embodiments the INSR antagonist comprises SEQ ID NO:1 and SEQ ID NO: 2, disulfides: SEQ ID NO:1 residue C6 and SEQ ID NO:1 residue C11; SEQ ID NO:1 residue C7 and SEQ ID NO:2 residue C7; SEQ ID NO:1 residue C20 and SEQ ID NO:2 residue C19.

In some embodiments the INSR antagonist comprises SEQ ID NO:3 and SEQ ID NO: 2, disulfides: SEQ ID NO:3 residue C6 and SEQ ID NO:3 residue C11; SEQ ID NO:3 residue C41 and SEQ ID NO:3 residue C48; SEQ ID NO:3 residue C7 and SEQ ID NO:2 residue C7, SEQ ID NO:3 residue C20 and SEQ ID NO:2 residue C19.

In some embodiments, a pharmaceutical formulation is provided, comprising an INSR antagonist as identified herein, and a pharmaceutically acceptable excipient. The formulation may be provided in a unit dose, e.g. a therapeutically effective dose. The formulation may be suitable for parenteral or topical delivery. The formulation may be provided in a lyophilized form, a sustained release form, a unit dose for direct administration, and the like as known in the art.

In methods of the disclosure, an effective dose of an INSR antagonist as identified herein is administered to an individual having, or at risk of having, hyperinsulinemia, in a dose effective to stabilize, reduce or prevent clinical symptoms of the disease. The individual may be monitored for clinical indicia of disease before, during, and/or after administration.

In addition to its effects on metabolic functions such as glucose transport, insulin can also stimulate the growth and proliferation of cancer cells, and insulin receptor antagonists can be used therapeutically to treat cancer. In some methods of the disclosure, an effective dose of an INSR antagonist as identified herein is administered to an individual having, or at risk of having, a condition in which insulin signaling is undesirable, including insulin-dependent cancer, in a dose effective to stabilize, reduce or prevent clinical symptoms of the disease. The individual may be monitored for clinical indicia of disease before, during, and/or after administration.

An INSR antagonist may be administered in a combination therapy. The effective dose of each drug in a combination therapy may be lower than the effective dose of the same drug in a monotherapy. In some embodiments the combined therapies are administered concurrently. In some embodiments the two therapies are phased, for example where one compound is initially provided as a single agent, e.g. as maintenance, and where the second compound is administered during a relapse, for example at or following the initiation of a relapse, at the peak of relapse, etc.

In an embodiment, methods are provided for producing an INSR antagonist of the disclosure. Peptides as disclosed herein may be synthesized by recombinant methods, or by solid phase peptide synthesis, as known in the art. One or more peptides may be ligated to form the final product, e.g. using Sortase A. Disulfide bonds may be formed through a stepwise process, or concomitantly by appropriate redox reactions.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1B. a. Human insulin (hIns) and synthetic insulin constructs equipped for A-Chain C-terminal modifications with varying linker lengths (1-2), SEQ ID NO:17, 18, 19 and SEQ ID NO:2. b. Sortase A conjugation scheme with model peptide (SEQ ID NO:20), GGGFYK.

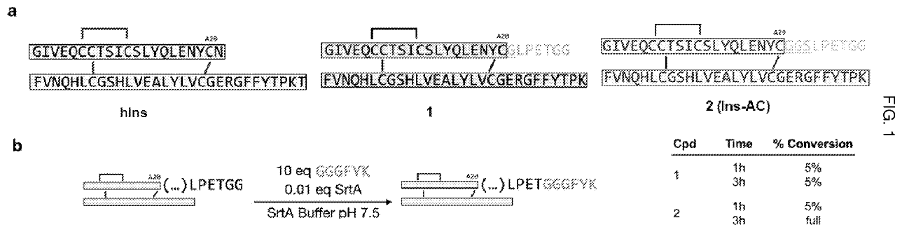

FIG. 2A-2C. Design and synthesis of the Ins-AC-based analogs. A. SEQ ID NO:21. B. SEQ ID NO:22 and 23. C. SEQ ID NO:24, 25, 26.

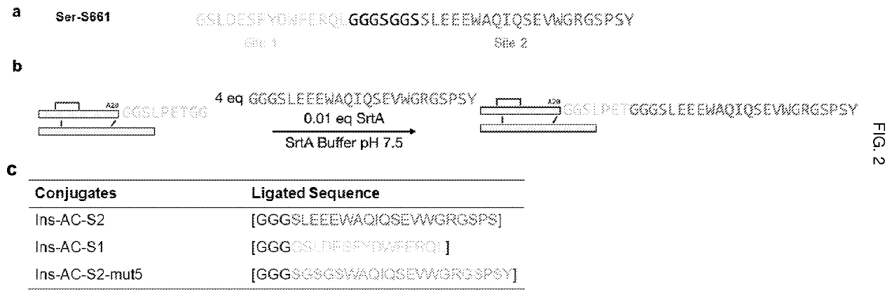

FIGS. 3A-3C. In vitro bioactivity assessments of Ins-AC analogs alone or in the presence of 43 nM native human insulin (hIns) in NIH 3T3 cells overexpressing human insulin receptor isoform A or B. a. In vitro pAKT assay measuring Ins-AC-S2-based agonism and antagonism in IR-B cells and b. IR-A cells. c. In vitro phospho-IR-beta assay measuring endogenous levels of phosphorylated insulin receptor-beta at Tyr1150/1151 when stimulated by Ins-AC-S2 with or without a constant concentration of hIns in IR-A cells. All the values are shown as mean±SD (n=4). EC50 and IC50 values were calculated by Prism 9 (GraphPad Software, California, USA) with nonlinear regression curve fitting of dose-response asymmetric equation.

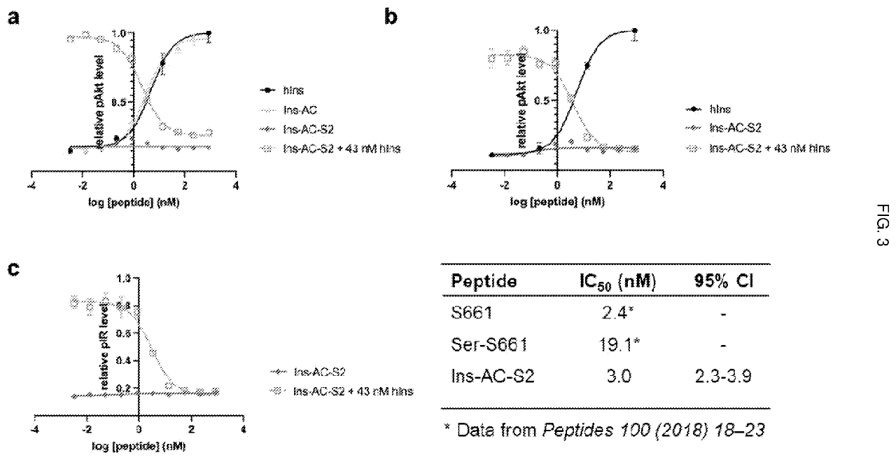

FIGS. 4A-4B. Exploring a. agonism and b. antagonism with additional Ins-AC-conjugates in NIH 3T3 cells overexpressing human insulin receptor isoform B. When the first five non-glycine amino acids in the oligoglycine sequence are removed, the subsequent Ins-AC-conjugate (Ins-AC-S2-des5) retains nearly full agonism and is unable to achieve antagonism at the insulin receptor. Ligating “Site 1” of the S661 peptide to the insulin construct (Ins-AC-S1) reveals that “Site 2” of S661 is essential for antagonistic activity. All the values are shown as mean±SD (n=4).

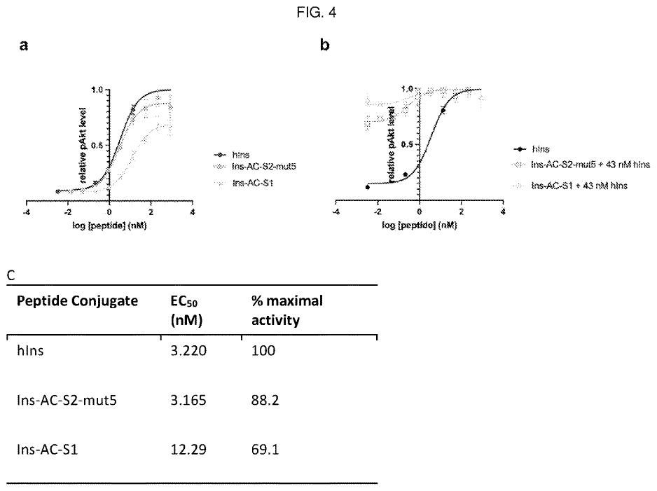

FIGS. 5A-5B. In vivo characterization of Ins-AC-S2 induced insulin-resistance in STZ-induced diabetic rats. a. Ins-AC-S2 treatment led to suppressed glucose lowering effect of insulin as determined by insulin tolerance test (ITT). (n=9) b. Ins-AC-S2 treatment led to significant decrease (p<0.01) in the maximum blood glucose change and significant increase (p<0.0001) in the AUC of the ITT in (a). Rats were all administered with 6 nmol/kg insulin after the pre-treatment of 50 nmol/kg Ins-AC-S2 or an equivalent vehicle control (PBS buffer). All the values are shown as mean±SD. ** P<0.01, **** P<0.0001 vs. vehicle control.

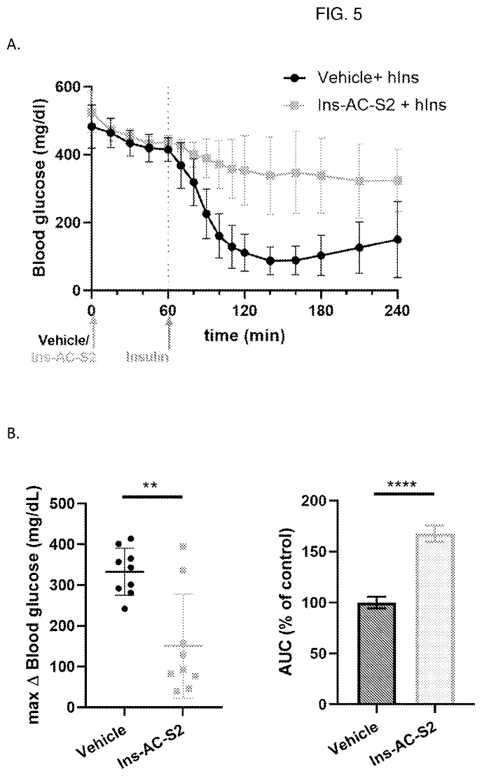

FIG. 6. Comparison of Ins-AC-S2 (SEQ ID NO:1 and SEQ ID NO:2) and Ins-AC-2-3 (SEQ ID NO:3 and SEQ ID NO:2).

FIG. 7. Activity of Ins-AC-2-27 polypeptide (SEQ ID NO:27 and SEQ ID NO:2) in pAKT assay.

FIG. 8. Activity of Ins-AC-6-5 polypeptide (SEQ ID NO:5 and SEQ ID NO:6) in pAKT assay.

FIG. 9. Activity of Ins-AC-2-7 (SEQ ID NO:7 and SEQ ID NO:2) in pAKT assay.

FIG. 10. Activity of Ins-AC-2-8 (SEQ ID NO:8 and SEQ ID NO:2) in pAKT assay.

FIG. 11 Activity of Ins-AC-10-9 in pAKT assay precursor structure (SEQ ID NO:9 and SEQ ID NO:10).

FIG. 12 Activity of Ins-AC-10-11 in pAKT assay (SEQ ID NO:11 and SEQ ID NO:10).

FIG. 13. Activity of Ins-AC-2-12, (SEQ ID NO:12 and SEQ ID NO:2).

FIG. 14. Activity of Ins-AC-2-3 (SEQ ID NO:3 and SEQ ID NO:2).

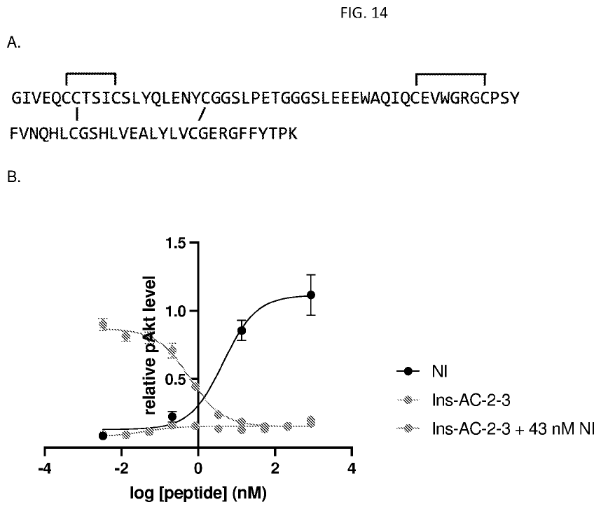

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The subject methods may be used for prophylactic or therapeutic purposes. As used herein, the term “treating” is used to refer to both prevention of relapses, and treatment of pre-existing conditions. For example, the prevention of hyperinsulinemia may be accomplished by administration of the agent prior to development of a relapse, after an initial diagnosis. “Treatment” as used herein covers any treatment of a disease in a mammal, particularly a human, and includes: inhibiting the disease symptom, i.e., arresting its development; or relieving the disease symptom, i.e., causing regression of the disease or symptom. The treatment of ongoing disease, where the treatment stabilizes or improves the clinical symptoms of the patient, is of particular interest.

“Inhibiting” the onset of a disorder shall mean either lessening the likelihood of the disorder's onset, or preventing the onset of the disorder entirely. Reducing the severity of a relapse shall mean that the clinical indicia associated with a relapse are less severe in the presence of the therapy than in an untreated disease. As used herein, onset may refer to a relapse in a patient that has ongoing relapsing remitting disease. The methods of the invention are specifically applied to patients that have been diagnosed with hyperinsulinemia.

“Diagnosis” as used herein generally includes determination of a subject's susceptibility to a disease or disorder, determination as to whether a subject is presently affected by a disease or disorder, prognosis of a subject affected by a disease or disorder (e.g., identification of disease states, stages, or responsiveness to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood, cerebral spinal fluid, and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “individual,” “subject,” “host,” and “patient,” used interchangeably herein and refer to any mammalian subject for whom diagnosis, treatment, or therapy is desired, for example humans, non-human primate, mouse, rat, guinea pig, rabbit, etc.

“Inhibiting” the expression of a activity of a receptor shall mean either lessening the degree to which receptor is activated, or preventing such activation entirely.

Prior art insulin receptor antagonists include XMetD, which is an allosteric monoclonal antibody to the insulin receptor (INSR) that was isolated from a human antibody phage display library. In addition to inhibiting the INSR via modulation of binding affinity, it also inhibited the INSR via modulation of signaling efficacy. XMetD binds similarly to both isoforms of the hINSR but minimally insulin-like growth factor-1 receptor (IGF-1R). The observations that insulin only partially inhibits XMetD binding to the hINSR and, reciprocally, that XMetD only partially inhibits insulin binding to the hINSR, are characteristic of negative allosteric modulation rather than competitive orthosteric inhibition. XMetD as well did not induce either INSR internalization or downregulation in the presence or absence of insulin. In contrast a positive control IgG, in the presence of insulin, did induce both INSR internalization and downregulation by greater than 50%. XMetD was also a negative modulator of hINSR activation. XMetD had no effect on INSR signaling in the absence of insulin. See Corbin et al. (2014) MAbs 6 (1): 262-72, herein specifically incorporated by reference.

Another allosteric mAb IR antagonist is IRAB-B. IRAB-B blocked insulin signaling within minutes in vitro and induced severe hyperglycemia as rapidly as 6 h in vivo. A single dose of IRAB-B also exhibited prolonged antagonist effects because significant decreases in insulin-induced IR phosphorylation were observed for at least 3 days in cells and severe hyperglycemia >575 mg/mL was measured in mice 2 weeks postinjection. XMetD, appears to be different from IRAB-B both in vitro and in vivo. In vitro, XMetD did not affect insulin-induced Akt phosphorylation at mAb levels of 13 nmol/L, whereas at least 10 nmol/L IRAB-B results in clear decreases in Akt phosphorylation. These differences between IRAB-B and XMetD may be due to different epitope engagement because both mAbs display low nanomolar binding affinity (IRAB-B K_{d} ~3 nmol/L and XMetD K_{d} 8 nmol/L).

Dosage and frequency of administration may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

An active agent can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants.

As noted above, an agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An agent can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds useful for administration with the active agents of the invention can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and from about one to about five amino acid modifications compared to the parent, and may have one, two three or more amino acid modifications.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

### Compositions

Insulin receptor antagonists of the disclosure are polypeptide heterodimers with a sequence and structure as set forth in Table 1. One or a cocktail of antagonists can be administered, e.g. 1, 2, 3 or more antagonists.

TABLE 1

| Name | Chain 1 | Chain 2 | Disulfide bonds |
|---|---|---|---|
| Ins-AC-S2 | SEQ ID NO: 1 | SEQ ID NO: 2 | SEQ ID NO: 1 residue C6 and SEQ ID NO: 1 residue C11 |
| | | | SEQ ID NO: 1 residue C7 and SEQ ID NO: 2 residue C7 |
| | | | SEQ ID NO: 1 residue C20 and SEQ ID NO: 2 residue C19 |
| Ins-AC-2-3 | SEQ ID NO: 3 | SEQ ID NO: 2 | SEQ ID NO: 3 residue C6 and SEQ ID NO: 3 residue C11 |
| | | | SEQ ID NO: 3 residue C41 and SEQ ID NO: 3 residue C48 |
| | | | SEQ ID NO: 3 residue C7 and SEQ ID NO: 2 residue C7 |
| | | | SEQ ID NO: 3 residue C20 and SEQ ID NO: 2 residue C19 |
| Ins-AC-2-27 | SEQ ID NO: 27 | SEQ ID NO: 2 | SEQ ID NO: 27 residue C6 and SEQ ID NO: 27 residue C11 |
| | | | SEQ ID NO: 27 residue C7 and SEQ ID NO: 2 residue C7 |
| | | | SEQ ID NO: 27 residue C20 and SEQ ID NO: 2 residue C19 |
| Ins-6-5 | SEQ ID NO: 5 | SEQ ID NO: 6 | SEQ ID NO: 5 residue C6 and SEQ ID NO: 5 residue C11 |
| | | | SEQ ID NO: 5 residue C7 and SEQ ID NO: 6 residue C25 |
| | | | SEQ ID NO: 5 residue C20 and SEQ ID NO: 6 residue C37 |
| Ins-AC-2-7 | SEQ ID NO: 7 | SEQ ID NO: 2 | SEQ ID NO: 7 residue C6 and SEQ ID NO: 7 residue C11 |
| | | | SEQ ID NO: 7 residue C7 and SEQ ID NO: 2 residue C7 |
| | | | SEQ ID NO: 7 residue C20 and SEQ ID NO: 2 residue C19 |
| Ins-AC-2-8 | SEQ ID NO: 8 | SEQ ID NO: 2 | SEQ ID NO: 8 residue C6 and SEQ ID NO: 8 residue C11 |
| | | | SEQ ID NO: 8 residue C7 and SEQ ID NO: 2 residue C7 |
| | | | SEQ ID NO: 8 residue C20 and SEQ ID NO: 2 residue C19 |
| Ins-AC-10-9 | SEQ ID NO: 9 | SEQ ID NO: 10 | SEQ ID NO: 9 residue C6 and SEQ ID NO: 9 residue C11 |
| | | | SEQ ID NO: 9 residue C7 and SEQ ID NO: 10 residue C7 |
| | | | SEQ ID NO: 9 residue C20 and SEQ ID NO: 10 residue C19 |
| Ins-AC-10-11 | SEQ ID NO: 11 | SEQ ID NO: 10 | SEQ ID NO: 11 residue C6 and SEQ ID NO: 11 residue C11 |
| | | | SEQ ID NO: 11 residue C7 and SEQ ID NO: 10 residue C7 |
| | | | SEQ ID NO: 11 residue C20 and SEQ ID NO: 10 residue C19 |
| Ins-AC-2-12 | SEQ ID NO: 12 | SEQ ID NO: 2 | SEQ ID NO: 12 residue C6 and SEQ ID NO: 12 residue C11 |
| | | | SEQ ID NO: 12 residue C7 and SEQ ID NO: 2 residue C7 |
| | | | SEQ ID NO: 12 residue C20 and SEQ ID NO: 2 residue C19 |

The sequences in the INSR antagonists are as set forth in Table 2:

TABLE 2

| | |
|---|---|
| SEQ ID NO: 1 | GIVEQCCTSICSLYQLENYCGGSLPETGGGSLEEEWAQIQSEVWGRGSPSY |
| SEQ ID NO: 2 | FVNQHLCGSHLVEALYLVCGERGFFYTPK |
| SEQ ID NO: 3 | GIVEQCCTSICSLYQLENYCGGSLPETGGGSLEEEWAQIQCEVWGRGCPSY |
| SEQ ID NO: 4 | GGGSLEEEEWAQIQSEVWGRGSPSY |
| SEQ ID NO: 5 | GIVEQCCTSICSLYQLENYCN |
| SEQ ID NO: 6 | SLEEEWAQIQSEVWGRGSFVNQHLCGSHLVEALYLVCGERGFFYTPKT |
| SEQ ID NO: 7 | GIVEQCCTSICSRYQLENYCGGSLPETGG |
| SEQ ID NO: 8 | GIVEQCCTSICSRYQLENYCGGSLPETGGGSLEEEWAQIQSEVWGRGSPSY |
| SEQ ID NO: 9 | GIVEQCCHRICSLYQLENYCGGSLPETGG |
| SEQ ID NO: 10 | FVNQHLCGSELVEALYLVCGERGFFYTPK |
| SEQ ID NO: 11 | GIVEQCCHRICSLYQLENYCGGSLPETGGGSLEEEWAQIQSEVWGRGSPSY |
| SEQ ID NO: 12 | GIVEQCCTSICSLYQLENYCGGSLPETGGGSLEEEWAQIQSEVWGRGSPDY |
| SEQ ID NO: 14 | FVNQHLCGSHLVEALYLVCGERGFFYTPKT |
| SEQ ID NO: 18 | GIVEQCCTSICSLYQLENYCGLPETGG |
| SEQ ID NO: 19 | GIVEQCCTSICLYQLENYCGGSLPETGG |
| SEQ ID NO: 20 | LPETGGGFYK |
| SEQ ID NO: 21 | GSLDESFYDWFERQLGGGSGGSGLEEEWAQIQSEVWGRGSPSY |
| SEQ ID NO: 22 | GGGSGLEEEWAQIQSEVWGRGSPSY |
| SEQ ID NO: 23 | GGSLPETGGGSLEEEWAQIQSEVWGRGSPSY |
| SEQ ID NO: 24 | GGGSLEEEWAQIQSEVWGRGSPS |
| SEQ ID NO: 25 | GGGGSLDESFYDWFERQL |
| SEQ ID NO: 26 | GGGSGSGSWAQIQSEVWGRGSPSY |
| SEQ ID NO: 27 | GIVEQCCTSICSLYQLENYCGGSLPETGGGSGGSGGSLEEEWAQIQSEVWGR |
| | GSPSY |
| SEQ ID NO: 28 | GSLDESFYDWFERQLGGGSGGSSLEEEWAQIQCEVWGRGCPSY |

The relative activity of the antagonists is provided in Table 3.

TABLE 3

| Name | Agonist Activity (nM) | Antagonist Activity (nM) |
|---|---|---|
| Ins-AC-S2 | NA | 2.085 |
| Ins-AC-2-3 | NA | 0.5936 |
| Ins-AC-2-27 | NA | 4.354 |
| Ins-6-5 | 0.3378 | NA |
| Ins-AC-2-7 | 21.63 | NA |
| Ins-AC-2-8 | NA | 5.473 |
| Ins-AC-10-9 | 0.5616 | NA |
| Ins-AC-10-11 | 0.01152 | 0.4997 |
| Ins-AC-2-12 | NA | 3.253 |

In some embodiments, the antagonist has an antagonistic activity of less than about 10 nM, less than about 5 nM, less than about 2.5 nM, less than about 1 nM.

In some embodiments, the antagonist can be conjugated to additional molecules to provide desired pharmacological properties such as extended half-life. In one embodiment, an antagonist can be fused to the Fc domain of IgG, albumin, or other molecules to extend its half-life, e.g. by pegylation, glycosylation, acylation with a fatty acid such as a C14 or C16 fatty acid, and the like as known in the art. In some embodiments the antagonist is conjugated to a polyethylene glycol molecules or “PEGylated.” The molecular weight of the PEG conjugated to the antagonist include but are not limited to PEGs having molecular weights between 5 kDa and 80 kDa, in some embodiments the PEG has a molecular weight of approximately 5 kDa, in some embodiments the PEG has a molecular weight of approximately 10 kDa, in some embodiments the PEG has a molecular weight of approximately 20 kDa, in some embodiments the PEG has a molecular weight of approximately 30 kDa, in some embodiments the PEG has a molecular weight of approximately 40 kDa, in some embodiments the PEG has a molecular weight of approximately 50 kDa, in some embodiments the PEG has a molecular weight of approximately 60 kDa in some embodiments the PEG has a molecular weight of approximately 80 kDa. In some embodiments, the molecular mass is from about 5 kDa to about 80 kDa, from about 5 kDa to about 60 kDa, from about 5 kDa to about 40 kDa, from about 5 kDa to about 20 kDa. The PEG conjugated to the polypeptide sequence may be linear or branched. The PEG may be attached directly to the orthogonal polypeptide or via a linker molecule. The processes and chemical reactions necessary to achieve PEGylation of biological compounds is well known in the art.

Antagonists can be acetylated at the N-terminus, using methods known in the art, e.g. by enzymatic reaction with N-terminal acetyltransferase and, for example, acetyl COA. The antagonist can be acetylated at one or more lysine residues, e.g. by enzymatic reaction with a lysine acetyltransferase. See, for example Choudhary et al. (2009). Science. 325 (5942): 834-840.

Fc-fusion can also endow alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The antagonist can include the entire Fc region, or a smaller portion that retains the ability to extend the circulating half-life of a chimeric polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases.

In other embodiments, an antagonist can comprise polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., Proc. Natl. Acad. Sci. USA 89:8145, 1992). In some embodiments, the antagonist further comprises a C-terminal c-myc epitope tag.

As described above, the antagonist of the invention may exist as a part of a chimeric polypeptide. In addition to, or in place of, the heterologous polypeptides described above, a polypeptide can contain sequences encoding a “marker” or “reporter.” Examples of marker or reporter genes include β-lactamase, chloramphenicol acetyltransferase (CAT), adenosine deaminase (ADA), aminoglycoside phosphotransferase (neo1, G418r), dihydrofolate reductase (DHFR), hygromycin-B-hosphotransferase (HPH), thymidine kinase (TK), lacz (encoding β-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter.

Antagonists may also include conservative modifications and substitutions at other positions of the polypeptide. Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: ala, pro, gly, gin, asn, ser, thr; Group II: cys, ser, tyr, thr; Group III: val, ile, leu, met, ala, phe; Group IV: lys, arg, his; Group V: phe, tyr, trp, his; and Group VI: asp, glu. In each instance, the introduction of additional modifications may be evaluated to minimize any increase in antigenicity of the modified polypeptide in the organism to which the modified polypeptide is to be administered.

The antagonists of the disclosure are incorporated into a variety of formulations for therapeutic administration. In one aspect, the antagonist is formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the active agents and/or other compounds can be achieved in various ways, usually by parenteral administration. The active agents and/or other compounds may be systemic after administration or may be localized by virtue of the formulation, or by the use of an implant that acts to retain the active dose at the site of implantation.

In pharmaceutical dosage forms, the active agents and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient and condition being treated and on the administration route, the active agent may be administered in dosages of 0.01 mg to 500 mg/kg body weight per day, e.g. about 20 mg/day for an average person. Dosages will be appropriately adjusted for pediatric formulation.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249:1527, 1990 and Hanes, Advanced Drug Delivery Reviews 28:97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

The antagonists provided by this disclosure have an affinity for IR sufficient to provide adequate binding for the intended purpose. Thus, for use as a therapeutic, the peptide, polypeptide, or protein provided by this invention should have an affinity (K_{d}) of between about 10⁻⁷ to about 10⁻¹⁵ M. More preferably the affinity is 10⁻⁸ to about 10⁻¹² M. Most preferably, the affinity is 10⁻¹⁰ to about 10⁻¹² M.

Also included within the scope of this invention are amino acid sequences containing substitutions, additions, or deletions based on the teachings disclosed herein and which bind to the insulin receptor with the same or altered affinity. For example, sequence tags (e.g., FLAG® tags) or amino acids, such as one or more lysines, can be added to the sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the consensus motifs described below, which comprise sequence tags (e.g., FLAG® tags), or which contain amino acid residues that are not associated with a strong preference for a particular amino acid, may optionally be deleted providing for truncated sequences. Certain amino acids (e.g., C-terminal or N-terminal residues) such as lysine which promote the stability or biotinylation of the amino acids sequences may be deleted depending on the use of the sequence, as for example, expression of the sequence as part of a larger sequence which is soluble, or linked to a solid support.

### Methods of Use

Insulin antagonists, also known as insulin receptor blockers, are pharmacological agents designed to counteract the effects of insulin in the body. These compounds play a crucial role in conditions where the modulation of insulin activity becomes necessary. One primary medical indication for administering an insulin antagonist is in the management of insulin overdose or hypoglycemic episodes. In cases where excessive insulin administration or endogenous insulin release leads to dangerously low blood glucose levels, the use of insulin antagonists can normalize blood sugar levels rapidly by blocking the actions of insulin receptors, thereby preventing further glucose uptake and utilization.

Insulin antagonists may be used in specific cases of hyperinsulinemia associated with insulinomas, pancreatic tumors that autonomously produce insulin. In such instances, the administration of insulin antagonists can help mitigate the hyperglycemic effects induced by excess insulin secretion. By competitively inhibiting insulin receptor activation, these antagonists contribute to the restoration of glucose balance and prevention of life-threatening hypoglycemic events.

The individual may be monitored for clinical indicia of disease before, during, and/or after administration. Methods of monitoring insulin levels and blood glucose are well known in the art, and may comprise monitoring blood glucose at defined periods of time, or may comprise continuous glucose monitoring using any of the commercially available patches, systems, etc. known and used for this purpose.

Congenital hyperinsulinism (CHI) is a heterogeneous and complex biochemical disorder which is characterized by the dysregulated release of insulin from pancreatic β-cell. In normal physiological state, the secretion of insulin is tightly coupled to glucose metabolism within the β-cell so that the insulin release is regulated to keep the plasma glucose concentration around 3.5-5.5 mmol/L. However, in CHI the secretion of insulin becomes unrelated to glucose metabolism, so that there is inappropriate insulin release for the plasma glucose level.

The genetic and molecular cause of CHI includes defects in key genes regulating insulin secretion from the pancreatic β-cell. Molecular defects in previously described genes (ABCC8, KCNJ11, GLUD1, GCK, HADH, SLC16A1, UCP2, HNF4A, HNF1A, HK1, PGM1, and PMM2) have been reported. However, recent studies have linked the role of other genes (CACNA1D, FOXA2) to hyperinsulinemic hypoglycaemia (HH) but in some of these cases the underlying molecular mechanisms are still not fully elucidated. Understanding the molecular mechanisms of CHI due to these genetic abnormalities has provided unique insight into the normal physiological mechanisms which regulate the insulin release.

Focal pancreatic adenomatous hyperplasia is seen in approximately 30%-40% of individuals, focal changes involve a limited region of the pancreas, with the remainder of the tissue being both histologically and functionally normal. A focal lesion is the confluence of apparently normal islets. Focal lesions typically are not macroscopically visible; they differ from true adenomas, which can be identified on gross inspection of the pancreas. Beta cells outside the focal lesion have small nuclei and sparse cytoplasm-histologic evidence that they are suppressed and not actively producing and secreting insulin.

Familial hyperinsulinism (FHI) is the most common cause of persistent neonatal hypoglycemia and should be considered in every infant presenting with unexplained hypoglycemia. Pathogenic variants in 14 genes have been associated with FHI. However, 40% of probands with FHI do not have an identified molecular cause. Causative genes include ABCC8; AD; CACNA1D; GCK; GLUD1; HADH; HK1; HNF1A; HNF4A; PMM2; KCNJ11; SLC16A1; UCP2. Broadly, these genetic defects leading to unregulated insulin secretion can be grouped into four main categories. The first group consists of defects in genes encoding the pancreatic K_{ATP} channels (ABCC8 and KCNJ11) and other channel/transporter proteins (KCNQ1, CACNA1D, SLC16A1). Pancreatic K_{ATP} channels have a critical role in the regulation of insulin release and defects in their encoding genes cause the most prevalent and severe forms of CHI. The second and third categories of conditions are enzymatic gene defects (GLUD1, GCK, HADH, UCP2, HK1, PMM2, PGM1) and defects in genes encoding the transcription factors (HNF1A, HNF4A, FOXA2) leading to changes in nutrient flux into metabolic pathways which converge on insulin secretion. The β-cell insulin release is coupled to the metabolic signals and so any perturbation in these pathways will ultimately result in inappropriate insulin secretion. Lastly, there are a large number of syndromic conditions (like Beckwith-Weidemann syndrome) which feature HH as a part of the syndrome while the underlying molecular mechanism leading to unregulated insulin secretion has yet to be clarified in most of the syndromes.

In combination with administration of an INSR antagonist of the disclosure, treatment may include administration of somatostatin analogs (e.g., octreotide or lanreotide) to suppress insulin secretion; nifedipine, which acts as an inhibitor of the voltage-dependent calcium channels present in the beta cell and inhibits insulin secretion; glucagon, which increases hepatic gluconeogenesis, glucocorticoids to induce resistance to endogenous insulin and correct the inadequate cortisol response. Individuals may also undergo dietary interventions, e.g. frequent high-carbohydrate feedings, including formula supplemented with glucose polymer.

Examples of doses for antagonists may include, but are not necessarily limited to a range from about 0.05 mg/kg to about 10 mg/kg (e.g., from about 0.1 mg/kg to about 10 mg/kg, from about 0.1 mg/kg to about 7.5 mg/kg, from about 0.1 mg/kg to about 5 mg/kg, from about 0.1 mg/kg to about 4 mg/kg, from about 0.1 mg/kg to about 3 mg/kg, from about 0.5 mg/kg to about 10 mg/kg, from about 0.5 mg/kg to about 7.5 mg/kg, from about 0.5 mg/kg to about 5 mg/kg, from about 0.5 mg/kg to about 4 mg/kg, from about 0.5 mg/kg to about 3 mg/kg, from about 1 mg/kg to about 10 mg/kg, from about 1 mg/kg to about 7.5 mg/kg, from about 1 mg/kg to about 5 mg/kg, from about 1 mg/kg to about 4 mg/kg, from about 1 mg/kg to about 3 mg/kg, about 1 mg/kg, about 2 mg/kg, about 3 mg/kg, about 4 mg/kg, about 5 mg/kg, about 7.5 mg/kg, or about 10 mg/kg).

Each of the active agents can be provided in a unit dose of from about 0.1 μg, 0.5 μg, 1 μg, 5 μg, 10 μg, 50 μg, 100 μg, 500 μg, 1 mg, 5 mg, 10 mg, 50, mg, 100 mg, 250 mg, 500 mg, 750 mg or more.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

The INSR antagonist may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length;

in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

General Information. All Fmoc amino acids, reagents, and solvents were used without purification. Fmoc amino acids were purchased from ChemPep and PurePep. Rink amide resin (catalog: 151801) and 2-chlorotrityl chloride (2-CTC) resin (catalog: 150301) were purchased from ChemPep. 1-[bis(dimethylamino)methylene]-1H-1,2,3-triazolo[4,5-b]pyridinium 3-oxid hexafluorophosphate (HATU) was purchased from Oakwood Chemical. N, N-dimethylformamide (DMF), dichloromethane (DCM), acetonitrile (MeCN), methanol (MeOH), diethyl ether (Et₂O), acetic anhydride (AC₂O), N,N-diisopropylethylamine (DIPEA), trifluoroacetic acid (TFA), 4-methylpiperidine, triisopropylsilane (TIPS), Dulbecco's phosphate buffer saline (PBS), ammonium bicarbonate (NH₄HCO₃), sodium hydroxide (NaOH), sodium chloride (NaCl), calcium chloride (CaCl₂)), and Tris-HCl were purchased from Fisher Scientific. Recombinant Sortase A5 protein (SrtA) was purchased from Active Motif. Native insulin was purchased from Invitrogen Life Technologies. Insulin building blocks were supplied by Novo Nordisk (Denmark).

Liquid Chromatography Mass Spectrometry (LC-MS) Analysis. Peptides were characterized using a Luna® 5 μm C18 100 Å (50×2 mm) column (Phenomenex, California, USA) on an Agilent 6120 Quadrupole LC-MS system at 0.4 mL/min beginning with 5% H₂O/MeCN (0.1% TFA) for 1 minute followed by a linear gradient from 5% to 95% H₂O/MeCN (0.1% TFA) over 5 minutes.

Reverse-Phase High Performance Liquid Chromatography (HPLC). Preparative reverse-phase HPLC of crude peptides was performed using a Luna® 5 μm C18 100 Å (250×21 mm) column (Phenomenex, California, USA) at 5 mL/min with a linear gradient from 20% to 50% H₂O/MeCN (0.1% TFA) over 30 minutes on an Agilent 1260 HPLC system at 220, 240, 260, and 280 nm. Fractions containing the desired product were lyophilized using a Labconco Freeze Dryer.

General Peptide Synthesis. Peptides were synthesized via Fmoc solid-phase peptide synthesis on Syro I (MultiSynTech GmbH) in a 10 mL reactor vial with a 0.1 mmol total loading capacity of the resin. The first C-terminal amino acid of the carboxylic acid C-terminus was coupled manually to 2-CTC resin: Fmoc-amino acid (0.1 mmol) and DIPEA (87.1 μL, 0.5 mmol) were dissolved in a solution of DMF and DCM (1:1, 2.5 mL). This solution was added to 2-CTC resin (250 mg) which was pre-washed 3×DMF and 3×DCM. After the reaction mixture rotated for 2 hours at room temperature, the resin was washed 3×DMF and 3×DCM and then capped with a solution of DCM, MeOH, and DIPEA (17:2:1, 5.0 mL) for 10 seconds 4 times. The resin was finally washed 3×DCM and 3×DMF. The first C-terminal amino acid of an amide C-terminus was coupled with Rink amide resin with the typical amino acid coupling conditions as follows: Fmoc was deprotected with 20% 4-methylpiperidine in DMF for 10 minutes twice at room temperature. Fmoc amino acids were coupled onto the resin with a solution of Fmoc-amino acid (0.5 mmol), HATU (0.5 mmol), and DIPEA (1.0 mmol) in DMF (2.5 mL) for 10 minutes at 50° C. (Cys and His) or at 70° C. (others). Arg residues were coupled twice under the same conditions at 70° C. The resin was washed 3×DMF between Fmoc deprotection and amino acid coupling.

Resin-bound peptides were washed 3×DMF and 3×DCM and dried under reduced pressure overnight. Peptides were cleaved from the resin with a solution of TFA, H₂O, and TIPS (38:1:1, 8 mL) after rotating for 2 hours at room temperature. Peptides were precipitated from the cleavage solution in cold Et₂O (80 mL). After centrifuging the peptides for 3 minutes at 3000×g, the supernatant was discarded, and the pellet of peptide was resuspended with Et₂O (40 mL). This was repeated 2 more times. The crude material was dried under reduced pressure overnight and checked via LC/MS.

Chain C, Insulin mimetic peptide S597. (see Park et al. (2022) Nat. Commun. 13 (1): 5594), (SEQ ID NO:2) Ac-SLEEEWAQIECEVYGRGCPSESFYDWFERQL-amide. Prior to cleavage, the resin-bound peptide was swelled with DMF for 10 minutes and DMF was removed by suction. A solution of Ac2O (94.5 μL, 1 mmol) and DIPEA (174 μL, 1 mmol) in DMF (4 mL) was added to the resin. A thefter reaction mixture rotated for 2 hours at room temperature, the solution was removed by suction and the resin was washing 3×DMF. After following the general cleavage protocol, the crude material was dissolved in 1% NH₄HCO₃ with 20% MeCN and stirred in an open-air environment for 24 hours. The oxidized product was purified on a Luna® 5 μm C18 100 Å (250×21 mm) column (Phenomenex) at 5 mL/min with a linear gradient from 20% to 60% of H₂O/MeCN (0.1% TFA) over 40 minutes on an Agilent 1260 HPLC system at 220, 240, 260, and 280 nm. The fractions containing S597 were flash frozen under liquid N2 and then lyophilized to yield the product as a white powder.

S661 is a high-affinity peptide antagonist to the insulin receptor, with the sequence, (SEQ ID NO:28) GSLDESFYDWFERQLGGGSGGSSLEEEWAQIQCEVWGRGCPSY-Amide (see Schäffer et al. (2008) Biochem Biophys Res Commun. 376 (2): 380-3). The crude material was dissolved in 1% NH₄HCO₃ with 20% MeCN and stirred in an open-air environment for 24 hours. The oxidized product was purified on a Luna® 5 μm C18 100 Å(250×21 mm) column (Phenomenex) at 5 mL/min with a linear gradient from 20% to 60% of H₂O/MeCN (0.1% TFA) over 40 minutes on an Agilent 1260 HPLC system at 220, 240, 260, and 280 nm. The fractions containing S661 were flash frozen under liquid N2 and then lyophilized to yield the product as a white powder.

Oligoglycine Peptide (SEQ ID NO:4) GGGSLEEEEWAQIQSEVWGRGSPSY. The crude product was dissolved in PBS (pH 7.5) with 20% MeCN and purified on a Luna® 5 μm C18 100 Å (250×21 mm) column (Phenomenex) at 5 mL/min with a linear gradient from 20% to 50% of H₂O/MeCN (0.1% TFA) over 30 minutes on an Agilent 1260 HPLC system at 220, 240, 260, and 280 nm. The fractions containing Oligoglycine Peptide were flash froze under liquid N2 and then lyophilized to yield the product as a white powder.

Antagonist Product Ins-AC-S2. (SEQ ID NO:1 and SEQ ID NO:2)

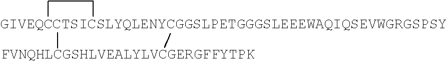

The reaction mixture was purified on a Luna® 5 μm C8 100 Å (250×10 mm) column (Phenomenex) at 2 mL/min with a linear gradient from 20% to 50% of H₂O/MeCN (0.1% TFA) over 40 minutes on an Agilent 1260 HPLC system at 220, 240, 260, and 280 nm. The fractions containing Antagonist Product were flash frozen under liquid N2 and lyophilized to give the product as a white powder.

Sortase A Ligation. 0.32 μmol of Novo 3, 4 equivalents of Oligoglycine Peptide, and 0.01 equivalents of SrtA (1 μg/μL) were mixed in 1 mL of sortase buffer (50 mM Tris-HCl, 5 mM CaCl₂, 150 mM NaCl, pH 7.5). After vigorous mixing, the solution reacted for 2.5 hours at room temperature. Antagonist product formation was confirmed via LC-MS.

Cell-based pAKT and phosphor-IR-beta Assays. The bioactivities of the analogs were evaluated through a cell-based pAKT (Ser473) assay or phospho-IR-beta (Tyr1150/1151) assay. Endogenous levels of pAKT or phosphor-IR-beta were measured in a human insulin receptor overexpressed NIH 3T3 cell line, derived from IGF-1R knockout mice (a generous gift from A. Morrione, Thomas Jefferson University). Cells were cultured in DMEM (Gibco) with 10% fetal bovine serum (FBS, Gibco), 100 U/mL penicillin-streptomycin (Gibco), 2 mg/mL puromycin (Thermo Fisher Scientific), and 1 mg/mL normocin (InvivoGen) at 37° C. under 5% CO₂. For the assays, 30,000 cells per well and 100 μl per well, were plated in a 96-well plate with culture media containing 1% FBS. 20 hours later, the media was removed and 50 μl of culture media with different concentrations of native insulin or analog was pipetted into each well. After treatment at 37° C. for 30 minutes, the solution was removed and the HTRF PAKT Ser473 kit or HTRF phosphor-IR-beta Tyr1150/1151 kit (Cisbio) was used to measure the phosphorylation of AKT or the insulin receptor. Briefly, the cells were first treated with cell lysis buffer (50 μl per well). After mild shaking for 1 hour, 16 μL of the cell lysate was added to 4 μl of the detecting reagent in a white 384-well plate. After 4 hours of incubation, the plate was read in a SpectraMax iD5 plate reader (Molecular Devices) and the data were processed according to the manufacturer's protocol. Normal insulin (NI) may be used as a control.

TABLE 4
MS Characterization of Analogs,

| Peptide | M/Z Expected (Da) | Observed M.W. (Da) | |
|---|---|---|---|
| S597 | 3823.7 | 1913.8 | [M/2 + 2H] |
| | | 1276.2 | [M/3 + 2H] |
| | | 957.4 | [M/4 + 2H] |
| S661 | 4798.1 | 1601.3 | [M/3 + 2H] |
| | | 1201.0 | [M/4 + 2H] |
| Oligoglycine Peptide | 2608.2 | 1305.2 | [M/2] |
| | | 870.8 | [M/3 + H] |
| Antagonist (SEQ ID NO: 1) | 8820.0 | 1261.7 | [M/7 + 4H] |
| | | 1104.1 | [M/8 + 4H] |

### Example 2

Ins-AC-S2 has the structure below (SEQ ID NO:1 and SEQ ID NO:2).

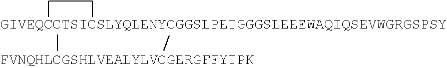

The analog Ins-AC-2-3 has the structure below (SEQ ID NO:3 and SEQ ID NO:2):

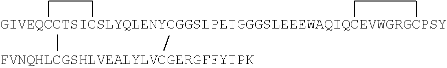

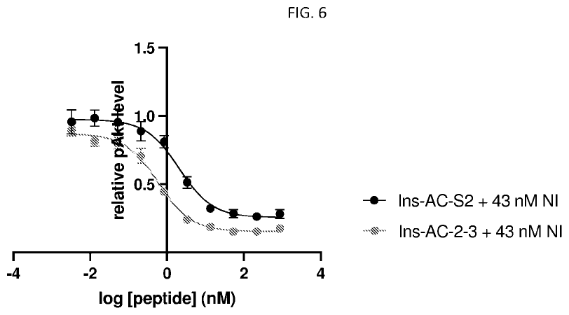

A comparison of the activity on the two molecules in the cell-based AKT assay described above is shown in FIG. 6, which shows that the disulfide S2 analog has the greater antagonistic activity.

The insulin core binds to site 1 of ∧-shape insulin receptor (IR). S2 sequence binds to site 2 of ∧-shape IR. The two compartments work together to hold the IR in the inactive A conformation as an antagonist. The insulin core does not bind to IR alone to activate the IR, so this analog has limited agonism. The S2 and overall binding are strong enough to provide antagonism but the binding between insulin core-S1 along, instead, is low enough to prevent the agonism.

The insulin core does not bind to IR alone, and the S2 is only served as a non-binding steric hindrance: Native hins binds firstly to site 1 of ∧-shape IR and then, or simultaneously with site 2, to initiate the IR conformational change to asymmetry. T-shape IR intermediate conformation, in which hIns fully contact with site 2 and slightly displace from site 1. Native hins further proceeds in an IR conformational change to activated T-shape IR, with full contact with site 1, and completely dissociated from site 2 contact. The S2 sequence is proposed to hijack the interaction between hins (insulin core) and site 2 of ∧-shape IR to prevent the initiation of conformational changing from ∧-shape IR to asym. T-shape IR intermediate. Consequently, the IR stay in ∧-shape and inactivated when binding to Ins-SA-S2. The S2 sequence has sufficient binding affinity to site 2 of ∧-shape IR during its proximity to site 2 while the insulin core binds to site 1 of ∧-shape IR. The linker length and flexibility are also important to simultaneously allow the insulin core to bind site 1 and the S2 sequence to reach the site 2 of ∧-shape IR. Extended length of linker increases the degree of freedom and might lose the sufficient proximity between S2 sequence and site 2, allowing the S2 sequence to escape from site 2 of ∧-shape IR and make insulin core activates IR alone.

### Example 3

### Ins-AC-2-27

Ins-AC-2-27 has the structure (SEQ ID NO:27 and SEQ ID NO:2)

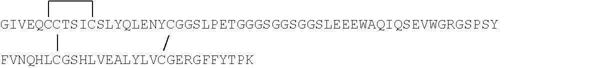

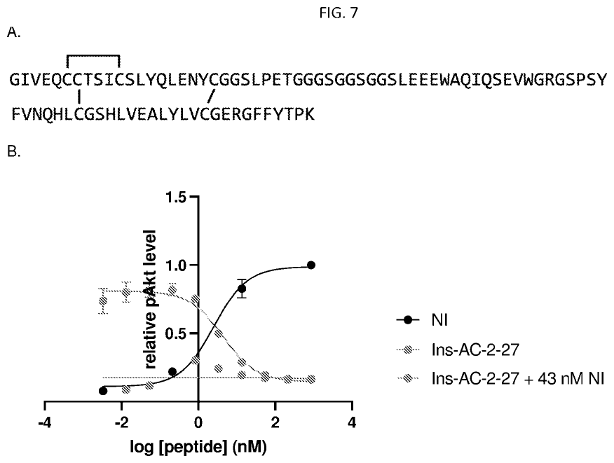

The additional SGGSGG spacer increases agonism. It may increase the degree of freedom to the orientation of insulin core and S2 sequence, which is less confined than the no linker Ins-AC-S2, and allows the S2 sequence to escape from the binding to site 2 of ∧-shape IR. The extra degree of freedom by additional SGGSGG space may result in a lower EC₅₀ than Ins-AC-S2. It provides more population of analog in lower concentrations. Activity is shown in FIG. 7.

### Example 4

### Ins-AC-6-5

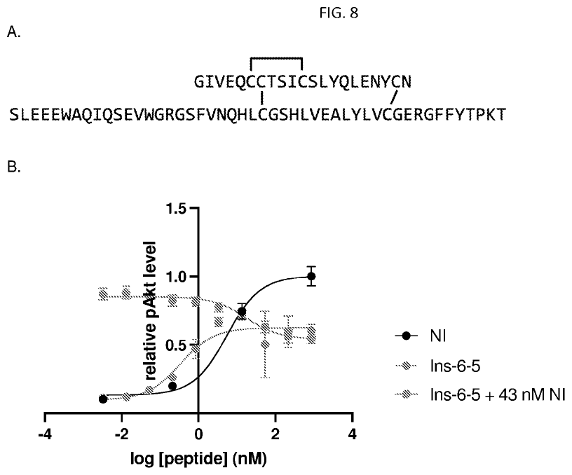

Ins-AC-6-5 has the structure (SEQ ID NO:5 and SEQ ID NO:6). Activity is shown in FIG. 8.

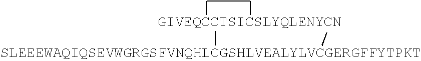

The B chain does not have the correct site 1-2 orientation of ∧-shape IR. It was expected to have a chance to match the site 1-2 orientation of ext. T-shape IR like S597 (PDB: 8dtl). The result appears that the S2 sequence is a steric hindrance of insulin core for agonism.

### Example 5

### Ins-AC-2-7

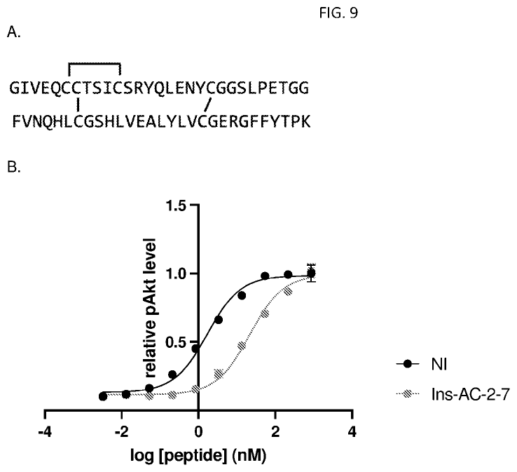

Ins-AC-2-7precursor has the structure (SEQ ID NO:7 and SEQ ID NO:2). Activity is shown in FIG. 9.

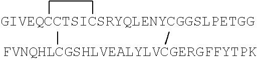

LeuA13R is a site 1 directed mutation, see for example (Nat. Struct. Mol. Biol. 2022, 29, 357-368).

### Example 6

### Ins-AC-2-8

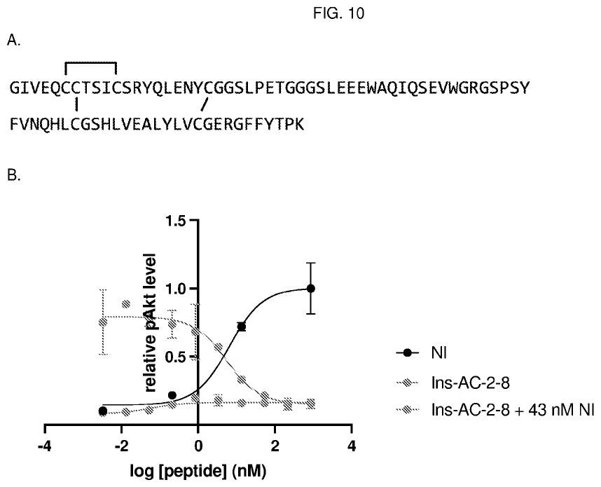

Ins-AC-2-8 has the structure (SEQ ID NO:8 and SEQ ID NO:2). Activity is shown in FIG. 10.

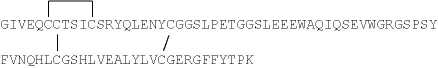

LeuA13R is a site 1-directed mutation (negative site 2 mutation). It may increase the binding specificity of insulin core to site 1 and also increase the chance of insulin core to bind site 1 alone (agonism). However, it may not increase the overall binding affinity. (Nat. Struct. Mol. Biol. 2022, 29, 357-368)

### Example 7

### Ins-AC-10-9

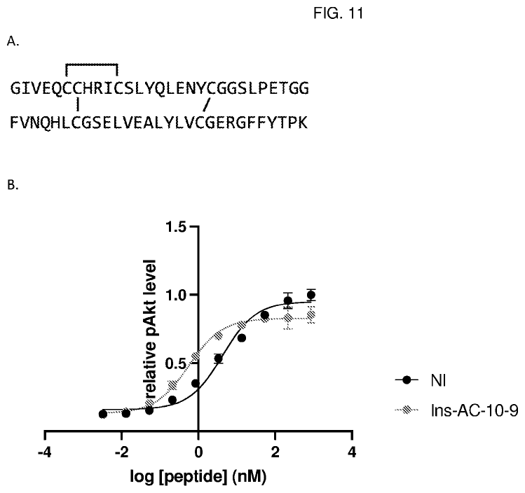

Ins-AC-10-9 has the structure (SEQ ID NO:9 and SEQ ID NO:10). Activity is shown in FIG. 11. The binding affinity is increased relative to the A13R mutation.

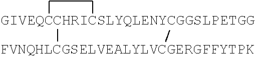

### Example 8

### Ins-AC-10-11

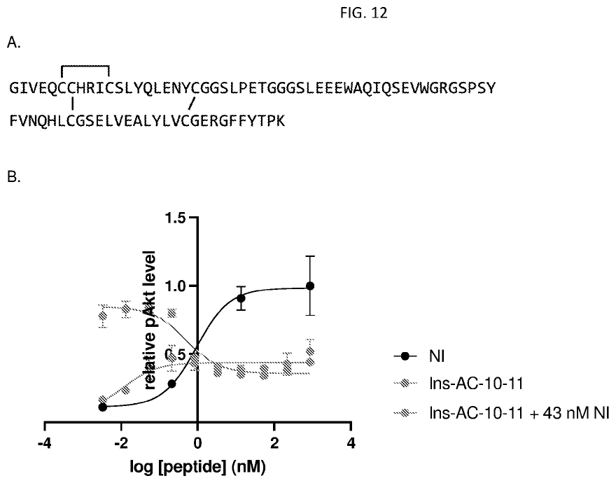

Ins-AC-10-11 has the structure (SEQ ID NO:11 and SEQ ID NO:10). Activity is shown in FIG. 12. The binding affinity is increased relative to the A13R mutation.

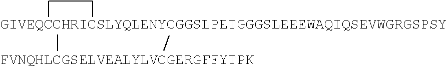

Similar reasons with the LeuA13R analog. Increasing site 1 affinity of insulin core may both increase overall binding (antagonism) and the insulin core binding along (agonism). In addition, HisB10Glu is also a site 2-directed mutation, which interacts with F1 site 2 and F1 site 1 (T-shape).

### Example 9

### Ins-AC-2-12

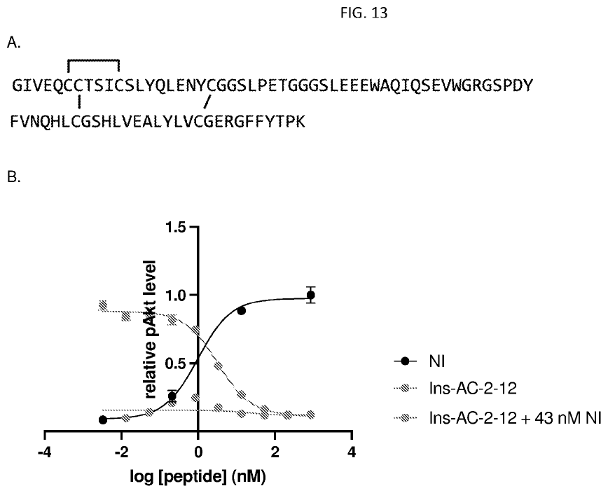

Ins-AC-2-12 has the structure (SEQ ID NO:12 and SEQ ID NO:2). Activity is shown in FIG. 13.

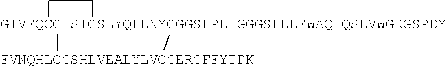

The S20D mutation interacts with site 1 of T-shape IR together with S1 sequence in S597. The S20D mutation may not have any improvement of site 2 affinity of ∧-shape IR (Nat. Commun. 2022, 13, 5594)

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. An insulin receptor (INSR) antagonist comprising a disulfide linked heterodimer of:
SEQ ID NO:1 and SEQ ID NO:2, disulfides: SEQ ID NO:1 residue C6 and SEQ ID NO:1 residue C11; SEQ ID NO:1 residue C7 and SEQ ID NO:2 residue C7; SEQ ID NO:1 residue C20 and SEQ ID NO:2 residue C19;
SEQ ID NO:3 and SEQ ID NO:2, disulfides: SEQ ID NO:3 residue C6 and SEQ ID NO:3 residue C11; SEQ ID NO:3 residue C41 and SEQ ID NO:3 residue C48; SEQ ID NO:3 residue C7 and SEQ ID NO:2 residue C7, SEQ ID NO:3 residue C20 and SEQ ID NO:2 residue C19;
SEQ ID NO:27 and SEQ ID NO:2, disulfides: SEQ ID NO:27 residue C6 and SEQ ID NO: 27 residue C11; SEQ ID NO:27 residue C7 and SEQ ID NO:2 residue C7; SEQ ID NO:27 residue C20 and SEQ ID NO:2 residue C19;
SEQ ID NO:5 and SEQ ID NO:6, disulfides: SEQ ID NO:5 residue C6 and SEQ ID NO:5 residue C11, SEQ ID NO:5 residue C7 and SEQ ID NO:6 residue C25, SEQ ID NO:5 residue C20 and SEQ ID NO:6 residue C37;
SEQ ID NO:7 and SEQ ID NO:2, disulfides: SEQ ID NO:7 residue C6 and SEQ ID NO:7 residue C11, SEQ ID NO:7 residue C7 and SEQ ID NO:2 residue C7, SEQ ID NO:7 residue C20 and SEQ ID NO:2 residue C19;
SEQ ID NO:8 and SEQ ID NO:2, disulfides: SEQ ID NO:8 residue C6 and SEQ ID NO:8 residue C11, SEQ ID NO:8 residue C7 and SEQ ID NO:2 residue C7, SEQ ID NO:8 residue C20 and SEQ ID NO:2 residue C19;
SEQ ID NO:9 and SEQ ID NO:10, disulfides: SEQ ID NO:9 residue C6 and SEQ ID NO:9 residue C11, SEQ ID NO:9 residue C7 and SEQ ID NO:10 residue C7, SEQ ID NO:9 residue C20 and SEQ ID NO:10 residue C19;
SEQ ID NO:11 and SEQ ID NO:10, disulfides: SEQ ID NO:11 residue C6 and SEQ ID NO: 11 residue C11, SEQ ID NO:11 residue C7 and SEQ ID NO:10 residue C7, SEQ ID NO:11 residue C20 and SEQ ID NO:10 residue C19; and
SEQ ID NO:12 and SEQ ID NO:2, disulfides: SEQ ID NO:12 residue C6 and SEQ ID NO: 12 residue C11, SEQ ID NO:12 residue C7 and SEQ ID NO:2 residue C7, SEQ ID NO:12 residue C20 and SEQ ID NO:2 residue C19.

2. The INSR antagonist of claim 1, comprising a disulfide linked heterodimer of SEQ ID NO:1 and SEQ ID NO:2.

3. The INSR antagonist of claim 1, comprising a disulfide linked heterodimer of SEQ ID NO:3 and SEQ ID NO:2

4. A pharmaceutical formulation comprising an INSR antagonist of claim 1.

5. The pharmaceutical formulation of claim 4, comprising a unit dose of antagonist.

6. The pharmaceutical formulation of claim 5, comprising a unit dose of from about from about 0.05 mg/kg to about 10 mg/kg of antagonist.

7. The pharmaceutical formulation of claim 4, formulated for parenteral administration.

8. The pharmaceutical formulation of claim 4, formulated for sustained release.

9. A method of treating a subject for a condition resulting in hyperinsulinemia, the method comprising:
administering an effective dose of a pharmaceutical formulation of claim 4 to a subject in need thereof.

10. A method of treating a subject for an insulin dependent cancer, the method comprising:
administering an effective dose of a pharmaceutical formulation of claim 4 to a subject in need thereof.

11. The method of claim 9, further comprising monitoring the individual for blood glucose following administration of the antagonist.

12. The method of claim 9, in combination with additional active agents.
